(19) United States
(12) Patent Application Publication
(10) Pub. No.: US 2023/0065778 A1
(43) Pub. Date: Mar. 2, 2023

(54) Occupancy Detection Using in-Bed Sensors

(71) Applicant: Apple Inc., Cupertino, CA (US)

(72) Inventors: Wegene H. Tadele, San Francisco, CA (US); Robert S. Parnell, San Jose, CA (US)

(21) Appl. No.: 17/900,370

(22) Filed: Aug. 31, 2022

Related U.S. Application Data
(60) Provisional application No. 63/239,218, filed on Aug. 31, 2021.

(51) Int. Cl. A61B 5/00; A61B 5/11

(52) U.S. Cl. CPC A61B 5/6891; A61B 5/1115; A61B 5/6892; A61B 5/0022; A61M 2205/50

(57) ABSTRACT

Embodiments of this disclosure are directed to an in-bed device having one or more substrates, a wireless transceiver, an antenna coupled to the wireless transceiver, and a control system. The one or more substrates are shaped to be positioned on a bed. The wireless transceiver is disposed on at least one of the one or more substrates, and configured to transmit and receive wireless pulses via the antenna. The control system is configured to cause the wireless transceiver to generate the wireless pulses and analyze the received wireless pulses to determine whether the bed is occupied by a user.

### CROSS-REFERENCE TO RELATED APPLICATION

This application is a nonprovisional of, and claims the benefit under 35 U.S.C. § 119(e) of and priority to U.S. Provisional Patent Application No. 63/239,218, filed Aug. 31, 2021, the contents of which are incorporated herein by reference in their entirety.

### FIELD

The described embodiments relate generally to in-bed sensors, or to a sensor system including such sensors. More particularly, the present embodiments relate to sensors capable of detecting bed occupancy based on reflective or transmissive detection (or non-detection) of electromagnetic radiation using one or more antennas.

### BACKGROUND

A variety of sensors may be used to monitor a user's sleep or health while the user is in bed (e.g., while the user is resting or sleeping). A variety of sensors may be similarly used to monitor a user's sleep or health while the user is resting or sleeping on other surfaces. In some cases, some in-bed sensors may be activated or deactivated in response to detecting whether a user occupies a bed. It is thus increasingly desirable to have reliable determination of bed occupancy, such that various in-bed sensors can be triggered for operation. These in-bed sensors can be used to detect various physiological parameters and conditions of the user.

### SUMMARY

The term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

Embodiments of this disclosure are directed to an in-bed device having one or more substrates, a wireless transceiver, an antenna coupled to the wireless transceiver, and a control system. The one or more substrates may be shaped to be positioned on a bed. The wireless transceiver may be disposed on at least one of the one or more substrates, and configured to transmit and receive wireless pulses via the antenna. The control system may be configured to cause the wireless transceiver to generate the wireless pulses and analyze the received wireless pulses to determine whether the bed is occupied by a user.

Embodiments of this disclosure are also directed to an in-bed device having a substrate, a wireless transmitter, a wireless receiver, a first antenna, a second antenna, and a control system. The substrate may be configured to be positioned on a bed. The wireless transmitter may be disposed at a first position on the substrate. The wireless receiver may be disposed at a second position on the substrate, where the second position is spaced apart from the first position. The first antenna may be coupled to the wireless transmitter. The wireless transmitter may be configured to transmit wireless pulses via the first antenna. The second antenna may be coupled to the wireless receiver. The wireless receiver may be configured to receive, via the second antenna, wireless pulses transmitted by the wireless transmitter. The control system may be configured to operate the wireless transmitter and the wireless receiver to, respectively, generate and receive the wireless pulses, and determine whether the bed is occupied by a user by analyzing the received wireless pulses.

Embodiments of this disclosure are also directed to a method of detecting bed occupancy. The method may include transmitting a first wireless pulse through a first antenna on an in-bed device and receiving the first wireless pulse. The method may further include decomposing the received first wireless pulse into amplitude and phase components. The method may also include determining, at least partly based on the amplitude and phase components, an occupancy condition of the bed.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims. Additional aspects of the disclosure will be apparent to those of ordinary skill in the art in view of the detailed description of various embodiments, which is made with reference to the drawings, a brief description of which is provided below.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 shows an example in-bed device that may be placed on a bed or other surfaces for detecting bed occupancy, according to certain aspects of the present disclosure;

FIG. 2A shows a top view of a first example in-bed device, according to certain aspects of the present disclosure;

FIG. 2B shows a cross-sectional side view of the example in-bed device of FIG. 2A, according to certain aspects of the present disclosure;

FIG. 3A shows a top view of a second example in-bed device, according to certain aspects of the present disclosure;

FIG. 3B shows a cross-sectional side view of the example in-bed device of FIG. 3A, according to certain aspects of the present disclosure; and

FIG. 4 shows a block diagram of a method of detecting bed occupancy, according to certain aspects of the present disclosure.

The use of cross-hatching or shading in the accompanying figures is generally provided to clarify the boundaries between adjacent elements and also to facilitate legibility of the figures. Accordingly, neither the presence nor the absence of cross-hatching or shading conveys or indicates any preference or requirement for particular materials, material properties, element proportions, element dimensions, commonalities of similarly illustrated elements, or any other characteristic, attribute, or property for any element illustrated in the accompanying figures.

The present disclosure is susceptible to various modifications and alternative forms, and some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

Additionally, it should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Various embodiments are described with reference to the attached figures, where like reference numerals are used throughout the figures to designate similar or equivalent elements. The figures are not necessarily drawn to scale and are provided merely to illustrate aspects and features of the present disclosure. Numerous specific details, relationships, and methods are set forth to provide a full understanding of certain aspects and features of the present disclosure, although one having ordinary skill in the relevant art will recognize that these aspects and features can be practiced without one or more of the specific details, with other relationships, or with other methods. In some instances, well-known structures or operations are not shown in detail for illustrative purposes. The various embodiments disclosed herein are not necessarily limited by the illustrated ordering of acts or events, as some acts may occur in different orders and/or concurrently with other acts or events. Furthermore, not all illustrated acts or events are necessarily required to implement certain aspects and features of the present disclosure.

For purposes of the present detailed description, unless specifically disclaimed, and where appropriate, the singular includes the plural and vice versa. The word “including” means “including without limitation.” Moreover, words of approximation, such as “about,” “almost,” “substantially,” “approximately,” and the like, can be used herein to mean “at,” “near,” “nearly at,” “within 3-5% of,” “within acceptable manufacturing tolerances of,” or any logical combination thereof. Similarly, terms “vertical” or “horizontal” are intended to additionally include “within 3-5% of” a vertical or horizontal orientation, respectively.

Additionally, directional terminology, such as “top”, “bottom”, “upper”, “lower”, “front”, “back”, “over”, “under”, “above”, “below”, “left”, “right”, etc. is used with reference to the orientation of some of the components in some of the figures described below. Because components in various embodiments can be positioned in a number of different orientations, directional terminology is used for purposes of illustration only and is in no way limiting. The directional terminology is intended to be construed broadly, and therefore should not be interpreted to preclude components being oriented in different ways. These words are intended to relate to the equivalent direction as depicted in a reference illustration; as understood contextually from the object(s) or element(s) being referenced, such as from a commonly used position for the object(s) or element(s); or as otherwise described herein. Further, it is noted that the term “signal” means a waveform (e.g., electrical, optical, magnetic, mechanical or electromagnetic) capable of traveling through a medium such as DC, AC, sinusoidal-wave, triangular-wave, square-wave, vibration, and the like.

Also, as used herein, the phrase “at least one of” preceding a series of items, with the term “and” or “or” to separate any of the items, modifies the list as a whole, rather than each member of the list. The phrase “at least one of” does not require selection of at least one of each item listed; rather, the phrase allows a meaning that includes at a minimum one of any of the items, and/or at a minimum one of any combination of the items, and/or at a minimum one of each of the items. By way of example, the phrases “at least one of A, B, and C” or “at least one of A, B, or C” each refer to only A, only B, or only C; any combination of A, B, and C; and/or one or more of each of A, B, and C. Similarly, it may be appreciated that an order of elements presented for a conjunctive or disjunctive list provided herein should not be construed as limiting the disclosure to only that order provided.

Embodiments of the disclosure relate to sensors capable of detecting bed occupancy based on reflective or transmissive detection (or non-detection) of electromagnetic radiation using one or more antennas, and to a sensor system including such sensors.

Bed occupancy detection is a useful component for sleep products (e.g., in-bed sensor mats). Similarly, presence detection can be useful for other products (e.g., to determine whether a user is sitting in a chair, sitting or lying on a couch or examination table, or otherwise present (or “on”) a surface). In-bed detection enables a sleep product to detect when a user is in-bed or out-of-bed, and this determination can then be used to trigger other sensors embedded in the mat (e.g., to turn the sensors on so that the sensors can collect data). Additionally, in-bed detection can be used to trigger operations by other devices (e.g., local or remote devices) that perform functions such as locking/unlocking a door, turning on/off music or a light, and so on. Some ways to perform in-bed detection involve using a long touch sensor or capacitive gap sensor to determine whether a user is lying or sitting on an in-bed sensor mat. However, these solutions can be relatively expensive since these types of sensors may need to span an entire dimension of a sleep product (e.g., an entire width or length of a mat or strip).

Another way to detect whether a user is in-bed or on a surface is to transmit and receive a wireless pulse, and analyze the receipt of the wireless pulse to identify the presence or absence of a user on a sleep product or other device. Analysis of the received wireless pulse may include decomposing the received wireless pulse into amplitude and phase (or imaginary and real impedance) components, to determine a loading or interference condition of one or more antennas.

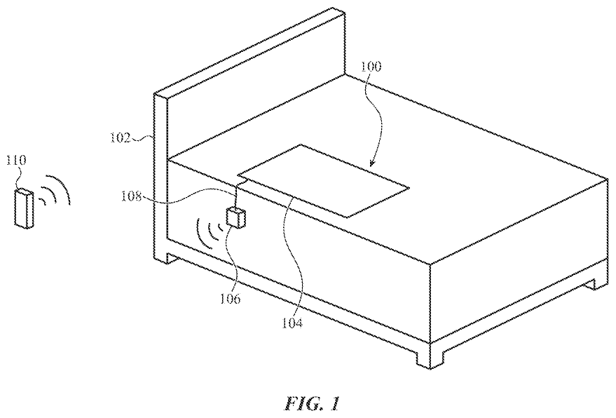

FIG. 1 shows an example in-bed device 100 that may be placed on a bed or other surfaces for detecting bed occupancy. The in-bed device 100 may be used to sense biological vibrations (e.g., chest cavity vibrations or sounds, nasal cavity vibrations or sounds, abdominal cavity vibrations or sounds, and the like) made by a person lying on a bed 102, a couch, a chair, an examination table, or the like. In addition to biological vibrations, the in-bed device 100 may sense other mechanical vibrations, pressures, forces, touches, displacements, temperatures, and so on. The in-bed device 100 may also or alternatively provide haptic feedback to a user who is lying or otherwise positioned on or near the in-bed device 100.

In some embodiments, the in-bed device 100 may include a sensing and/or haptic feedback mat 104 (or strip). Although the mat 104 is shown to have a rectangular shape (and in some cases may have a square shape), the mat 104 may alternatively have any shape, including an elliptical shape (which in some cases may be a circular shape). Electronic, optical, pneumatic, hydraulic, and/or other types of processing, and control circuits may be included within the mat 104 or in a dongle 106 attached to the mat 104. A sheath 108 may enclose wires, cables, flex circuits, optical fibers, air or liquid passageways (e.g., tubes), or other interconnects that extend between the mat 104 and the dongle 106. Additionally or alternatively, some or all of these types of interconnects may be routed within the mat 104. In the case of pneumatic or hydraulic elements, the dongle 106 (or alternatively the mat 104) may house a pneumatic or hydraulic pump.

Sensors that may be housed within the mat 104 include pressure sensors, temperature sensors, touch sensors, displacement sensors, force sensors, and the like. In some cases, pressure sensors may include an array of pressure sensing pockets or bladders that sense pressure pneumatically (or hydraulically). In some cases, a temperature sensor may be a resistive temperature sensor. In some cases, a touch sensor or displacement sensor may include a set of capacitive electrodes. In some cases, a force sensor may include a set of capacitive electrodes or a strain sensor. Other types of sensors, and other implementations of pressure, temperature, touch, displacement, or force sensors, are also contemplated.

Haptic feedback devices may also be housed within the mat 104. In some cases, one or more pockets or bladders of a pneumatic or hydraulic sensor can also be housed within the mat 104 for providing haptic feedback. Piezoelectric actuators, capacitive or electrostatic electrodes, and other types of haptic feedback devices may also be housed within the mat 104. Pneumatic, vibratory, or other types of feedback may be provided by the haptic feedback devices.

The mat 104 and the sensors and haptic feedback devices included therein may be flexible and/or deformable, so that they are more or less unnoticeable to a person lying on the bed 102 until they are actuated to provide haptic feedback to the person.

The processing and control circuits housed within the dongle 106 or the mat 104 may provide electrical, optical, pneumatic, hydraulic, or other types of control signals to the various sensors or haptic feedback devices housed within the mat 104. The processing and control circuits may also receive sensor measurements, and in some cases may receive haptic feedback measurements, and may process, analyze, or convey the measurements. In some embodiments, the processing and control circuits may amplify and digitize signals for further processing. In some cases, the processing and control circuits may include a communications interface for communicating digitized signals or other information to a remote device 110, such as a smartphone or an electronic watch. The communications interface may also receive instructions from the remote device 110. For example, the communications interface may receive instructions, control signals, settings, or queries from the remote device 110. The communications interface may be wireless (e.g., a WiFi or BLUETOOTH® interface) or wired (e.g., a universal serial bus (USB) interface). In some cases, the processing and control circuitry may include a processor (e.g., a microprocessor, a microcontroller, an application-specific integrated circuit (ASIC), or a field-programmable gate array (FPGA)).

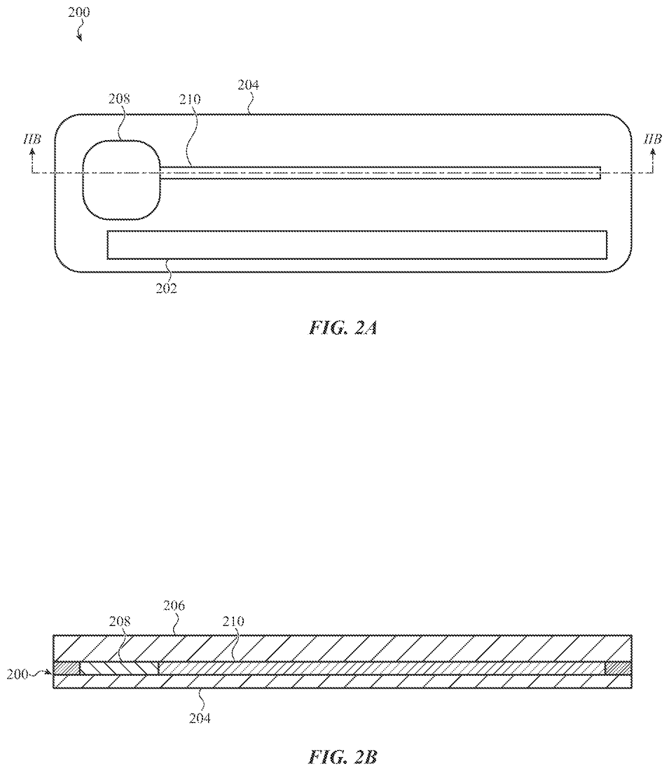

FIG. 2A shows a top view of a first example in-bed device 200 that incorporates reflective detection (or non-detection) of electromagnetic radiation, using a single antenna therein. FIG. 2B shows a cross-sectional side view of the example in-bed device 200, formed along the lines IIB of FIG. 2A. The in-bed device 200 is substantially similar to the in-bed device 100, described with respect to FIG. 1 above. The in-bed device 200 may take the form of a mat or strip, which may be configured as described with reference to FIG. 1. The in-bed device 200 may include various sensors and/or haptic devices, processing and control circuits, and so on, as described with reference to FIG. 1, and as generally identified as a control system 202 in FIG. 2A. The in-bed device 200 includes a substrate 204 (e.g., a flex circuit) on which the control system 202 is mounted. The substrate 204 is shaped to be positioned on a bed. In some embodiments, the in-bed device 200 may include sensors, processing and control circuits, and/or other components mounted on, and distributed between, two or more substrates. In some embodiments, part of the control system 202 may be moved to a dongle or remote device that is electrically or wirelessly coupled to the in-bed device 200, as described with reference to FIG. 1.

The in-bed device 200 is positioned in or under a mat 206 formed from a bed fabric that is flexible and/or deformable. In some embodiments, the mat 206 may be inserted in a pouch made of fabric, polymer (e.g., plastic), silicone rubber, or other material and suited for positioning on a bed or under a user. In some embodiments, the substrate 204, a wireless transceiver 208, and an antenna 210 coupled to the wireless transceiver 208 may be housed within the pouch. In some embodiments, a haptic feedback device may be housed within the mat 206. The haptic feedback device may include one or more pockets or bladders of a pneumatic or hydraulic sensor for providing haptic feedback. In some embodiments, piezoelectric actuators, capacitive or electrostatic electrodes, and other types of haptic feedback devices may also be housed within the mat 206.

The in-bed device 200 further includes the wireless transceiver 208 (e.g., a radio) disposed on the substrate 204. The wireless transceiver 208 transmits and receives wireless pulses via the antenna 210 coupled to the wireless transceiver 208. In some embodiments, the antenna 210 may include one or more conductive traces disposed on or in the substrate 204, or on or in a flex circuit, wires, or cables coupled to the wireless transceiver 208 and/or substrate 204. In some embodiments, the wireless transceiver 208 may be positioned toward one end of the in-bed device 200 (e.g., a left end, a right end, etc.). In some embodiments, the antenna 210 may span a substantial portion of the in-bed device 200 (e.g., more than 50%, 75%, or 90% of a width or length of the in-bed device 200).

In some embodiments, the wireless transceiver 208 may be a WiFi or ultra-wideband (UWB) transceiver. In some cases, the wireless transceiver may simultaneously transmit a current wireless pulse while receiving a reflection (or reflections) of one or more previously transmitted wireless pulses. A WiFi wireless transceiver may in some cases operate in the range of 2 GHz-5 GHz. A UWB wireless transceiver may in some cases operate in the range of 5 GHz-8 GHz. The wireless transceiver 208 may alternatively be any other form of radio frequency (RF) or microwave transceiver.

The control system 202 includes processing and control circuitry that are configured to cause the wireless transceiver to generate the wireless pulses (e.g., by operating the wireless transceiver 208). Reflections of the wireless pulses received by the wireless transceiver 208 are then analyzed by the control system 202 to determine whether a bed including the in-bed device 200 is occupied or not occupied by a user. The analysis of the received reflections may include, for example, decomposing the received reflections into amplitude and phase (or imaginary and real impedance) components, to determine a loading or interference condition of the antenna 210.

In some embodiments, in response to detecting that the bed is occupied by the user, the control system 202 may turn on a sensor configured to capture a parameter (e.g., a physiological parameter such a blood pressure, heart rate) of the user. In some embodiments, in response to detecting that the bed is occupied by the user, the control system 202 may apply a haptic feedback to the user, through the haptic feedback devices described above. In some embodiments, in response to detecting that the bed in not occupied by the user, the control system 202 may turn off the sensor in the in-bed device 200 or transition the sensor to a low-power state.

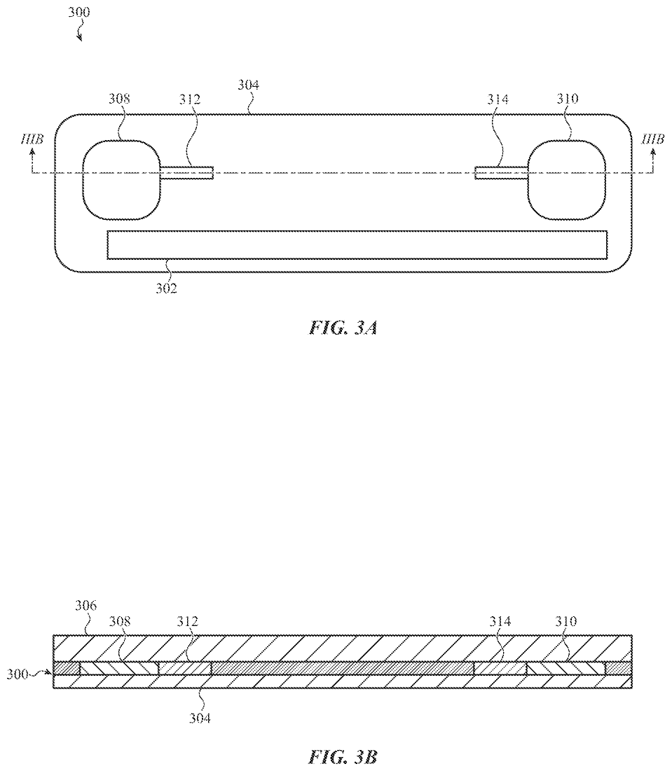

FIG. 3A shows a top view of a second example in-bed device 300 that incorporates transmissive detection (or non-detection) of electromagnetic radiation using a pair of antennas therein. FIG. 3B shows a cross-sectional side view of the example in-bed device 300, formed along the lines IIIB of FIG. 3A. The in-bed device 300 is substantially similar to the in-bed device 100, described with respect to FIG. 1 above. The in-bed device 300 may take the form of a mat or strip, which may be configured as described with reference to FIG. 1. The in-bed device 300 may include various sensors and/or haptic devices, processing and control circuits, and so on, as described with reference to FIG. 1, and generally identified as a control system 302 in FIG. 3A. The in-bed device 300 includes a substrate 304 (e.g., a flex circuit) on which the control system 302 is mounted. The substrate 304 is shaped to be positioned on a bed. In some embodiments, the in-bed device 300 may include sensors, processing and control circuitry, and/or other components mounted on, and distributed between, two or more substrates. In some embodiments, part of the control system 302 may be moved to a dongle or remote device that is electrically or wirelessly coupled to the in-bed device 300, as described with reference to FIG. 1.

The in-bed device 300 is positioned in or under a mat 306 (or alternatively, bed sheet or other bed fabric), and in some embodiments, may be inserted in a pouch made of fabric, polymer (e.g., plastic), silicone rubber, or other material that is flexible and/or deformable and suited for positioning on a bed or under a person. In some embodiments, the substrate 304, a wireless transmitter 308, a wireless receiver 310, and respective antennas 312, 314 may be housed within the pouch. In some embodiments, a haptic feedback device may be housed within the mat 306. The haptic feedback device may include one or more pockets or bladders of a pneumatic or hydraulic sensor for providing haptic feedback. In some embodiments, piezoelectric actuators, capacitive or electrostatic electrodes, and other types of haptic feedback devices may also be housed within the mat 306.

The in-bed device 300 further includes the wireless transmitter 308 (or in alternate embodiments, a first wireless transceiver 308) disposed at a first position on the substrate 304, and the wireless receiver 310 (or in alternate embodiments, a second wireless transceiver 310) disposed at a second position on the substrate 304, spaced apart from the first position. In the alternate embodiments, the first wireless transceiver 308 may be a first radio, while the second wireless transceiver 310 may be a second radio. In alternate embodiments, the first wireless transceiver 308 may include a wireless transmitter and the second wireless transceiver 310 may include a wireless receiver.

The wireless transmitter 308 may transmit (and in the alternate embodiments, also receive) wireless pulses via a first antenna 312 coupled to the wireless transmitter 308. The wireless receiver 310 may receive (and in the alternate embodiments, also transmit) wireless pulses via a second antenna 314 coupled to the wireless receiver 310. In some embodiments, the first antenna 312 and the second antenna 314 may extend towards one another. In some embodiments, the first antenna 312 and the second antenna 314 are linear antennas that extend along a common line. In some embodiments, the wireless transmitter 308 may be positioned toward a first end of the in-bed device 300 and the wireless receiver 310 may be positioned toward a second end of the in-bed device 300 (e.g., opposite the first end).

In some embodiments, the first antenna 312 and the second antenna 314 may include one or more conductive traces disposed on or in the substrate 304, or on or in a flex circuit, wires, or cables coupled to the wireless transmitter 308, the wireless receiver 310, and/or the substrate 304. The wireless pulses received through the second antenna 314 may be the same wireless pulses transmitted by the wireless transmitter 308 or those transmitted from elsewhere. In some embodiments, the wireless transmitter 308 (or in alternate embodiments, the first wireless transceiver 308) and the wireless receiver 310 (or in alternate embodiments, the second wireless transceiver 310) may each be a WiFi device, a UWB device, or alternatively, any other form of RF or microwave transmitters or receivers.

The control system 302 includes processing and control circuitry that are configured to operate the wireless transmitter and wireless receiver to, respectively, generate and receive the wireless pulses (or in alternative embodiments, operate the first wireless transceiver 308). The control system 302 may then determine whether a bed including the in-bed device 300 is occupied (or not occupied) by a user, by analyzing the wireless pulses received by the wireless receiver 310 or the second wireless transceiver 310. The analysis of the received wireless pulses may include, for example, decomposing the received wireless pulses into amplitude and phase (or imaginary and real impedance) components, to determine a loading or interference condition of the antennas 312, 314.

In some embodiments, in response to detecting that the bed is occupied by the user, the control system 302 may turn on a sensor configured to capture a parameter (e.g., a physiological parameter such as blood pressure, heart rate) of the user. In some embodiments, in response to detecting that the bed is occupied by the user, the control system 302 may apply a haptic feedback to the user, through the haptic feedback devices described above. In some embodiments, in response to detecting that the bed in not occupied by the user, the control system 302 may turn off the sensor in the in-bed device 300 or transition the sensor to a low-power state.

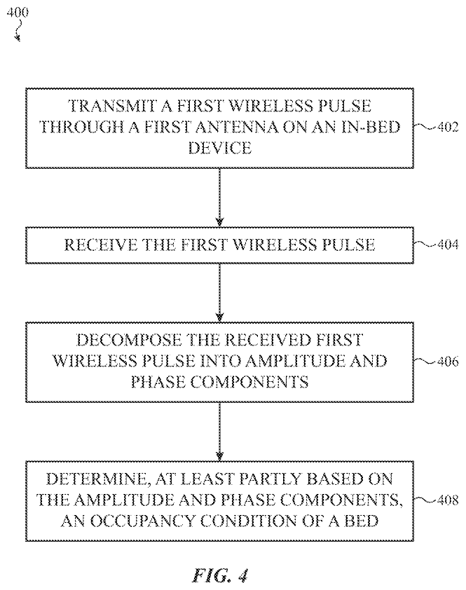

FIG. 4 shows a block diagram 400 of an example method of detecting bed occupancy using the in-bed devices 200, 300 described above. The method begins in block 402, where a first wireless pulse is transmitted through a first antenna on an in-bed device. In some embodiments, where the in-bed device includes a single wireless transceiver to which the first antenna is coupled, the wireless transceiver may be positioned on one side of the in-bed device and operate at a frequency between about 2 GHz and about 8 GHz. In some embodiments, where the in-bed device includes two different wireless transceivers (or alternatively, a wireless transmitter and a wireless receiver) each having their respective antennas, the two wireless transceivers may be spaced apart at two different positions on the in-bed device and operate at a frequency between about 2 GHz and about 8 GHz.

In block 404, the first wireless pulse is received. In some embodiments where the in-bed device includes a single wireless transceiver, the first wireless pulse is received through a reflection thereof along the first antenna, termed a “reflective detection” approach. In other embodiments, where the in-bed device includes two different wireless transceivers (or alternatively, a wireless transmitter and a wireless receiver) each having their respective antennas, termed a “transmissive detection” approach, the first wireless pulse is received at a second antenna coupled to the receiving wireless transceiver (or alternatively, the wireless receiver) that is spaced apart from the first antenna coupled to the transmitting wireless transceiver (or alternatively, the wireless transmitter).

In block 406, the received first wireless pulse is decomposed into amplitude and phase components. This can be accomplished through decomposition techniques that are commonly known in signal processing. The decomposition into amplitude and phase components may include determining a loading or interference condition of the first antenna and/or the second antenna.

In block 408, an occupancy condition of the bed is determined, at least partly based on the amplitude and phase components. In some embodiments, in response to detecting that the bed is occupied by the user, a sensor may be turned on configured to capture a parameter (e.g., a physiological parameter such as blood pressure, heart rate) of a user occupying the bed. In some embodiments, in response to detecting that the bed is occupied by the user, a haptic feedback may be applied to the user, through the haptic feedback devices included in the in-bed devices. In some embodiments, in response to detecting that the bed in not occupied by the user, the sensor in the in-bed devices may be turned off or transitioned to a low-power state.

The foregoing description, for purposes of explanation, uses specific nomenclature to provide a thorough understanding of the described embodiments. However, it will be apparent to one skilled in the art, after reading this description, that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art, after reading this description, that many modifications and variations are possible in view of the above teachings.

As described above, one aspect of the present technology may be the gathering and use of data available from various sources, including biometric data. The present disclosure contemplates that, in some instances, this gathered data may include personal information data that uniquely identifies or can be used to identify, locate, or contact a specific person. Such personal information data can include, for example, biometric data and data linked thereto (e.g., demographic data, location-based data, telephone numbers, email addresses, home addresses, data or records relating to a user's health or level of fitness (e.g., vital signs measurements, medication information, exercise information), date of birth, or any other identifying or personal information).

The present disclosure recognizes that the use of such personal information data, in the present technology, can be used to the benefit of users. For example, the personal information data can be used to authenticate a user to access their device, or gather performance metrics for the user's interaction with an augmented or virtual world. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services or anytime thereafter. In another example, users can select not to provide data to targeted content delivery services. In yet another example, users can select to limit the length of time data is maintained or entirely prohibit the development of a baseline profile for the user. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

Although the disclosed embodiments have been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

While various embodiments of the present disclosure have been described above, it should be understood that they have been presented by way of example only, and not limitation. Numerous changes to the disclosed embodiments can be made in accordance with the disclosure herein, without departing from the spirit or scope of the disclosure. Thus, the breadth and scope of the present disclosure should not be limited by any of the above described embodiments. Rather, the scope of the disclosure should be defined in accordance with the following claims and their equivalents.

What is claimed is:

1. An in-bed device, comprising:
one or more substrates shaped to be positioned on a bed;
a wireless transceiver disposed on at least one of the one or more substrates;
an antenna coupled to the wireless transceiver, the wireless transceiver configured to transmit and receive wireless pulses via the antenna; and
a control system configured to,
cause the wireless transceiver to generate the wireless pulses; and
analyze the received wireless pulses to determine whether the bed is occupied by a user.

2. The in-bed device of claim 1, further comprising:
a deformable mat in which the one or more substrates, the wireless transceiver, and the antenna are housed; and
a haptic feedback device housed within the deformable mat; wherein,
the control system is configured to,
determine, before actuating the haptic feedback device to provide haptic feedback to the user, the bed is occupied by the user.

3. The in-bed device of claim 2, wherein the haptic feedback device comprises a pneumatic feedback device.

4. The in-bed device of claim 1, wherein the antenna further comprises one or more conductive traces disposed on at least one substrate of the one or more substrates.

5. The in-bed device of claim 1, wherein the wireless transceiver is positioned on one side of the in-bed device and the antenna spans at least 75% of a width of the in-bed device.

6. The in-bed device of claim 1, wherein the wireless transceiver operates at a frequency between 2 GHz and 8 GHz.

7. The in-bed device of claim 1, further comprising a pouch made of fabric, plastic, or silicone rubber, wherein the one or more substrates, the wireless transceiver, and the antenna are housed in the pouch.

8. An in-bed device, comprising:
a substrate configured to be positioned on a bed;
a wireless transmitter disposed at a first position on the substrate;
a wireless receiver disposed at a second position on the substrate, the second position spaced apart from the first position;
a first antenna coupled to the wireless transmitter, the wireless transmitter configured to transmit wireless pulses via the first antenna;
a second antenna coupled to the wireless receiver, the wireless receiver configured to receive, via the second antenna, the wireless pulses transmitted by the wireless transmitter; and
a control system configured to,
operate the wireless transmitter and the wireless receiver to, respectively, generate and receive the wireless pulses; and
determine whether the bed is occupied by a user by analyzing the received wireless pulses.

9. The in-bed device of claim 8, wherein the first antenna and the second antenna extend toward each other.

10. The in-bed device of claim 9, wherein the first antenna and the second antenna are linear antennas and extend along a common line.

11. The in-bed device of claim 8, wherein the first antenna and the second antenna each further comprise one or more conductive traces disposed on the substrate.

12. The in-bed device of claim 8, wherein the wireless transmitter and the wireless receiver are ultra-wideband transceivers.

13. The in-bed device of claim 8, wherein the wireless transmitter and the wireless receiver are Wi-Fi transceivers.

14. The in-bed device of claim 8, further comprising:
a first wireless transceiver including the wireless transmitter; and
a second wireless transceiver including the wireless receiver.

15. A method, comprising:
transmitting a first wireless pulse through a first antenna on an in-bed device;
receiving the first wireless pulse;
decomposing the received first wireless pulse into amplitude and phase components; and
determining, at least partly based on the amplitude and phase components, an occupancy condition of a bed.

16. The method of claim 15, wherein receiving the first wireless pulse comprises receiving a reflection of the first wireless pulse along a first antenna.

17. The method of claim 15, wherein receiving the first wireless pulse comprises receiving the first wireless pulse at a second antenna spaced apart from the first antenna.

18. The method of claim 17, further comprising:
determining the bed is not occupied; and
powering a sensor of the in-bed device off or transitioning the sensor to a low-power state in response to determining the bed is not occupied.

19. The method of claim 15, further comprising:
determining the bed is occupied by a user; and
powering on a sensor to capture a parameter of the user, in response to determining the bed is occupied by the user.

20. The method of claim 15, further comprising:
determining the bed is occupied by a user; and
applying haptic feedback to the user in response to determining the bed is occupied by the user.
